(19) United States
(12) Patent Application Publication
(10) Pub. No.: US 2024/0335351 A1
(43) Pub. Date: Oct. 10, 2024

(54) AN INGESTIBLE CAPSULE DEVICE WITH ONE OR MORE MOTION DEVICES FOR GASTROINTESTINAL TRACT STIMULATION

(71) Applicant: KNOCKEAT LTD., Haifa (IL)

(72) Inventors: Roni SHABAT, Kfar Yehezkel (IL); Itamar DACH, Haifa (IL)

(21) Appl. No.: 18/292,923

(22) PCT Filed: Jul. 21, 2022
(86) PCT No.: PCT/IL2022/050786

§ 371 Date: Jan. 28, 2024

Related U.S. Application Data
(60) Provisional application No. 63/203,728, filed on Jul. 29, 2021.

(51) Int. Cl. A61H 23/02; A61H 21/00

(52) U.S. Cl. CPC A61H 23/0263; A61H 21/00; A61H 2023/0272; A61H 2201/1223; A61H 2201/1666; A61H 2201/1676; A61H 2201/169; A61H 2201/501; A61H 2201/5058; A61H 2201/5097

(57) ABSTRACT

An ingestible capsule device (ICD) comprising at least: a capsule-housing; and at least one motion device. Each motion device is located inside the ICD and has a power supply, a fixture element, at least one motor, and at least one weight assembly movable by use of the at least one motor, wherein the weight assembly is movably connected, via a hinge, to the fixture element to allow pendulum-like swinging movement of the weight assembly, in respect to the fixture element, about a swinging axis “x”. The swinging movement about the swinging axis “x” causes pounding of the weight of the weight assembly over at least one inner wall of the capsule-housing. The motion device may be configured to stimulate a gastrointestinal tract (GTI) section of the subject, by transmitting pounding forces of the weight assembly from the inner wall of the capsule-housing onto an inner wall of the GIT section.

### CROSS REFERENCES TO RELATED APPLICATIONS

This application is a national phase entry of International Patent Application No. PCT/IL/2022/050786, filed Jul. 21, 2022, which claims the benefit under 35 U.S.C. § 119 (b) to U.S. Provisional Patent Application No. 63/203,728, filed Jul. 29, 2021, both of which are hereby incorporated by reference.

### TECHNICAL FIELD

The present disclosure relates in general to ingestible capsules having an operable device therein and more particularly to ingestible capsules having a device therein for gastrointestinal-tract (GIT) stimuli.

### BACKGROUND

Gastrointestinal (GI) peristalsis is a coordinated contraction and relaxation of muscles along one or more parts of the alimentary canal such as sections of the small and/or large intestines, induced by one or more GI reflexes such as the gastroileal reflex.

Constipation or gastrointestinal impaction can be caused due to various reasons such as malfunctioning of the GI muscles, hormonal problems, unhealthy and fiber-poor diet or a combination of several causes. Some individuals suffer from severe GI pain when defecating due to scars, wounds, sores etc. caused due to a surgical procedure or natural causes such as autoimmune diseases, bowel polyps, and the like. These situations require immediate pain alleviation by induction of GI peristaltic movement, diluting, softening, reducing acidity level of chyme or feces in the GI tract, etc.

Equine colic is a relatively common disorder in horses and is a common equine death-cause. Equine colic is typically treated by administration of lubrication materials (such as oils) for bowl lubrication, administration of analgesic medication for pain relief and/or nasogastric intubation. In some cases, e.g., cases in which a bowel-twisting or looping is suspected, surgical intervention is required in order to surgically remove bowl-impactions and save the horse's life.

### BRIEF DESCRIPTION OF THE FIGURES

The figures illustrate generally, by way of example, but not by way of limitation, various embodiments discussed in the present document.

For simplicity and clarity of illustration, elements shown in the figures have not necessarily been drawn to scale. For example, the dimensions of some of the elements may be exaggerated relative to other elements for clarity of presentation. Furthermore, reference numerals may be repeated among the figures to indicate corresponding or analogous elements. References to previously presented elements are implied without necessarily further citing the drawing or description in which they appear. The figures are listed below.

FIGS. 1A-1D show an ingestible capsule device (IC) having a motion device, according to some embodiments: FIG. 1A shows a transparent view of the ICD; FIG. 1B shows a pendulum like motion device of the ICD for causing a weight assembly thereof to swing with respect to a swinging axis; FIG. 1C shows a weight assembly of the motion device; and FIG. 1D shows the motion device where its weight assembly is in a swinging position.

FIGS. 2A-2D show an ingestible capsule device (ICD) having a motion device, according to other embodiments: FIG. 2A shows a non-transparent view of the ICD; FIG. 2B shows a motion device of the ICD; FIG. 2C shows the motion device having a weight assembly thereof in a tilted-swinging position; FIG. 2D shows the weight assembly in an approximated view;

FIGS. 3A-3B show an ingestible capsule device (ICD) having a motion device with an additional weight, according to yet other embodiments: FIG. 3A shows a transparent view of the ICD; and FIG. 3B shows the motion device of the ICD;

FIGS. 4A-4B show an ingestible capsule device (ICD) having a motion device with an additional weight, according to yet other embodiments: FIG. 4A shows a transparent view of the ICD; and FIG. 4B shows the motion device of the ICD;

FIG. 5 shows optional configuration of a programmable processing and control module (PPCM) operable by a control unit of one or more motion devices of the ICD, according to some embodiments;

FIG. 6 shows a motion device for the ICD, having a weight assembly movably connectable to the fixture element via a hinge, in which the hinge is directly rotatable by the motor, by having a drive shaft of the hinge attached to the hinge and the weight assembly fixedly connected to the hinge, according to some embodiments;

FIGS. 7A-7B show an ingestible capsule device (ICD) having a motion device with two weight assemblies, each rotatable about a different hinge, where each of the weight assemblies includes a separate eccentric weight element, according to additional or alternative embodiments: FIG. 7A shows a transparent view of the ICD; and FIG. 7B shows the motion device;

FIG. 8 shows an ingestible capsule device (ICD) having a motion device with two weight assemblies one having an eccentric weight element and the other does not include an eccentric weight element, each rotatable about a different hinge, according to additional or alternative embodiments; and

FIG. 9 shows an ingestible capsule device (ICD) having at least one magnetic sensor, for identification of one or more magnetic activation sequences, according to some embodiments, by use of an external magnetic instrument having at least two magnetic poles.

### DETAILED DESCRIPTION

Aspects of disclosed embodiments pertain to ingestible capsule devices, motion devices and methods for stimulating inner wall sections of a gastrointestinal tract of subjects.

The term gastrointestinal tract (GIT) may refer to the tract (tubular organ(s)) of the digestive system of a subject, from the mouth to the anus, including for example, the mouth cavity, the gullet, the stomach, the small intestines and the large intestines.

According to some embodiments, the ingestible capsule device (ICD) may include, for example, at least: a capsule-housing; and at least one motion device, where the motion device(s) of the specific ICD is (are) located inside the capsule-housing.

The dimensions of the ICD may be such as to enable a subject (Depending on animal size and GIT size and dimensions) to swallow the ICD for ingestion thereof, therefore may require using miniature size components for the motion devices located inside the capsule-housing of the ICD.

According to some embodiments, the motion device may be configured to generate pounding movement of a weight, on the capsule-housing's inner wall, for transmittal of the pounding movements to the inner wall of the GIT of the subject.

The term “pounding” or any inflection thereof may refer to one or more of: knocking, hammering, hitting, striking, banging, beating, battering, etc.

The pounding movements may be repeatable, cyclic and/or arbitrary in time and intensity.

According to some embodiments, the motion device may include at least:
(i) a fixture element;
(ii) at least one motor setup including at least one motor;
(iii) a weight assembly including at least one weight element, movable by use of the at least one motor setup; and
a power supply such as one or more batteries, for powering the at least one motor setup;

According to some embodiments, the weight assembly may be movably connected, via a hinge, to the fixture element to allow pendulum-like swinging movement of the weight assembly, with respect to the fixture element, about a swinging axis.

According to some embodiments, the swinging movement about the swinging axis may causes pounding or knocking of the weight assembly on an inner wall of the capsule-housing, where the stimulation of a section of the GTI of the subject may be caused by having the pounding forces of the weight assembly against the inner wall of the capsule-housing transmitted onto an inner wall of the GIT section.

The term “subject” or “individual” mentioned herein may refer to any living being having a digestive system, such as any mammal such as a human, a domesticated farm animal such as a horse, a livestock (domesticated) animal such as a cow, a sheep, a goat, etc.

The term “ingesting” or “ingestible” or any other inflections thereof, used herein, may refer to the act of swallowing of a capsule such as of the ingestible capsule devices.

The terms “capsule” or “ingestible capsule” may be used interchangeably herein and may relate to any object/device in the size, shape, external surface texture and material composition that can be swallowed by subjects such as in the shape of an oval or spherical pill.

According to some embodiments, the capsule-housing of the ICD may be made from a material that cannot be dissolved or eroded during its entire contact with the subject's GIT or any other organs and parts of the subject, to prevent the content of the ingestible capsule device (mainly the motion device located inside the capsule-housing) from being exposed to body liquids as well as to prevent damage that may be caused to body tissue by the contents of the ingestible capsule device.

According to some embodiments, the weight assembly of the motion device may further include a rotatable element configured to be rotated by the at least one motor setup, around a rotation axis, which is angular, e.g., perpendicular to the swinging axis). The rotation speed and direction (clockwise and counterclockwise in relation to the rotation axis) may be controllable and changeable.

According to some embodiments, the at least one weight element of the weight assembly may be connected to the rotatable element in an off-axis location on the rotatable element, with respect to the rotation axis, where the off-axis location of the at least one weight element may cause changes in the center of mass with respect to the swinging axis when the rotatable element is rotated, thereby causing the pendulum-like swinging movement about the swinging axis.

According to some embodiments, the ICD may further include a control unit such as a printed circuit board (PCB), a control chip, one or more switching devices, one or more sensors, one or more transmitters, one or more receivers, one or more processors, a micro-electro-mechanical system (MEMS), and the like, for controlling operation of the at least one motor setup.

According to some embodiments, the maximal length of the at least one motor setup may range between 4-10 mm.

According to some embodiments, the control unit may be further configured to receive operation command signals and operate the at least one motor setup according to received operation command signals.

According to some embodiments, the ICD may further include at least one sensor located therein or on an external surface of the capsule-housing, for example, for sensing one or more of: one or more characteristics of the ingestible capsule device; time from ingestible capsule device intake; location of the ingestible capsule device; one or more characteristics of an environment of the ingestible capsule device.

According to some embodiments, the control unit may also include a programmable processing and control unit (PPCU). The PPCU may be configured at least to one or more of:
control operation of the at least one motor based on a programmable operation plan;
receive sensor data from the at least one sensor;
analyze the received sensor data to determine one or more characteristics of the ingestible capsule device and/or an environment thereof;
control operation of the ingestible capsule device, based on determined one or more characteristics of the ingestible capsule device and/or an environment of the ingestible capsule device.

According to some embodiments the motor(s) used by each motor set of each motion device of the ICD may be controlled by controlling rotation speed, operation frequency, e.g., by controlling electrical current/power rate/level, and/or current/power supply frequency of change (e.g., frequency of on/off switching, power/current level reduction and increasing frequency etc.).

The frequency of the pendulum-like swinging movement of the weight assembly with respect to the fixture element may be adjustable by adjusting power/current frequency, duration, and power level and may enable adapting the swinging frequency to the specific subject type (determined by subject's weight ranges, animal type, age etc.) or to the specific subject (depending on the specific colic subject disorder or problem).

According to some embodiments, the ingestible capsule device is operated by controlling electrical current/power supply to each of the at least one motor setup in one or more manners such as:
intermittently changing motor setup current/power supply for intermittent change in motor setup activation and/or operation mode;
activating the at least one motor setup at a specific timing after the at least motor setup is turned on enabling activation after an activation delay period for allowing the ingestible capsule device to pass specific sections of the GIT before being activated or stop its activity before a specific location in the GIT;
remote and/or external activation and operation control via a remote control device controllable and operable by a user;
generating occasion random activation of the at least one motor;
generating random changes in activation power and duration of the at least one motor.

Operating the motion device at random operation characteristics such as random motor power changes (causing random pounding force applied to the GIT inner wall) may be used to prevent/reduce the GIT of the subject from developing tolerance to the stimulation applied thereto.

According to some embodiments, the at least one motor setup may include at least one of: an electrical motor, a micro or miniature direct current (DC) motor, a micro alternating current (AC) motor a cylindrical rotating micro motor. The motor may be configured to enable switching rotation/swinging sides of the weight assembly in a controllable speed e.g., by switching current directions using the AC motor.

According to some embodiments, the weight assembly may include two or more weight elements: an internal weight element embedded within the at least one motor setup and at least one external weight element configured to add an additional weight to the overall weight of the weight assembly.

According to some embodiments, the motion device may include several motion devices all located and operated within the same capsule-housing, where each motion device is operated by a separate motor setup using the same or separate power supplies. The swinging axes of the several motion devices of the same ICD may be: all parallel to one another; coaxial; all angular (non-parallel) to one another; or one or more of the several swinging axes being parallel to one or more other swinging axes while one or more are angular to at least one other motion device.

According to some embodiments, the at least one weight assembly may connect to an inner wall of the capsule-housing via an elastic connecting element, such as a spring coil, enabling retraction of the weight assembly to a relaxed state.

The capsule-housing in general may be made from biocompatible material(s) that do not decompose or deteriorate in the GIT of the subject and can withstand the pressure in the GIT such as various types of medical polymers such as in polycarbonates.

According to some embodiments, at least one part of the capsule-housing, where the weight assembly engages the inner wall of the capsule-housing when pounding thereover, is made from a flexible or elastic material and/or of a lower thickness than the thickness of other parts of the capsule-housing, for enhancing transmission of pounding forces from the capsule-housing's inner wall to the inner wall of the subject's GIT section.

For example, the first part of the capsule-housing, which does not include the weight assembly, may be thicker in width than the second part of the capsule-housing which includes the weight assembly.

According to some embodiments, all motion devices of the same ICD may share and use the same power supply, or each may use a different power supply.

According to some embodiments, the operation of the multiple motion devices of the same ICD may be done in a coordinated, programmable, adjustable and/or planned manner and optionally with occasional random operation for GTI tolerance reduction/prevention.

According to some embodiments, the motion device of each ICD may be programmed to operate on occasions with equal or changing break-time-intervals in which the motion device is not operated e.g., to avoid/prevent GIT tolerance.

The maximal or minimal size of the entire ICD may depend on the size of the subject type or age. For example, the maximal size (length/width) of ICDs designated for horses for treating equine colic may be significantly larger than the overall maximal IDS length/width designated for dogs or human subjects.

According to some embodiments, the ICD may also be activated within the stomach part of the GIT for stomach stimulation as well as for large and small intestinal stimulation.

According to some embodiments, the control unit may be configured to gradually increase motor power, rotation speed and/or swinging frequency, once activated, to enable it to reach its maximal pounding abilities once passed the esophagus or the stomach.

According to some embodiments, the activation of the ICD may be made by use of a bipolar magnet or a magnet instrument having two magnets of opposite polarities, each magnet attached to a different opposite edge of a holding stick, where the control unit of the ICD is configured to automatically activate the ICD when receiving a preset code of a sequence, performed within a short time-span (such as within a 20 seconds) of magnetic poles being used. For example, the sequence may include: +-+poles requiring a user to flip the magnet device such that its positive pole first faces the ICD, then the negative pole and then the positive pole again all within 20 seconds, to avoid misreading other magnetic forces applied to the ICD during its use. The control unit may include at least one magnetic sensor, for sensing variations in the magnetic field, where the processing means of the control unit may be configured to receive sensor signal and identify the performed code/sequence, and activate the ICD upon code identification.

According to some embodiments, the control unit may be programmed to include several different operation modes (each associated with a different operation program) each associated with a different activation code/sequence such that the same ICD batch may be used in different manners for different subjects and their varied medical requirements.

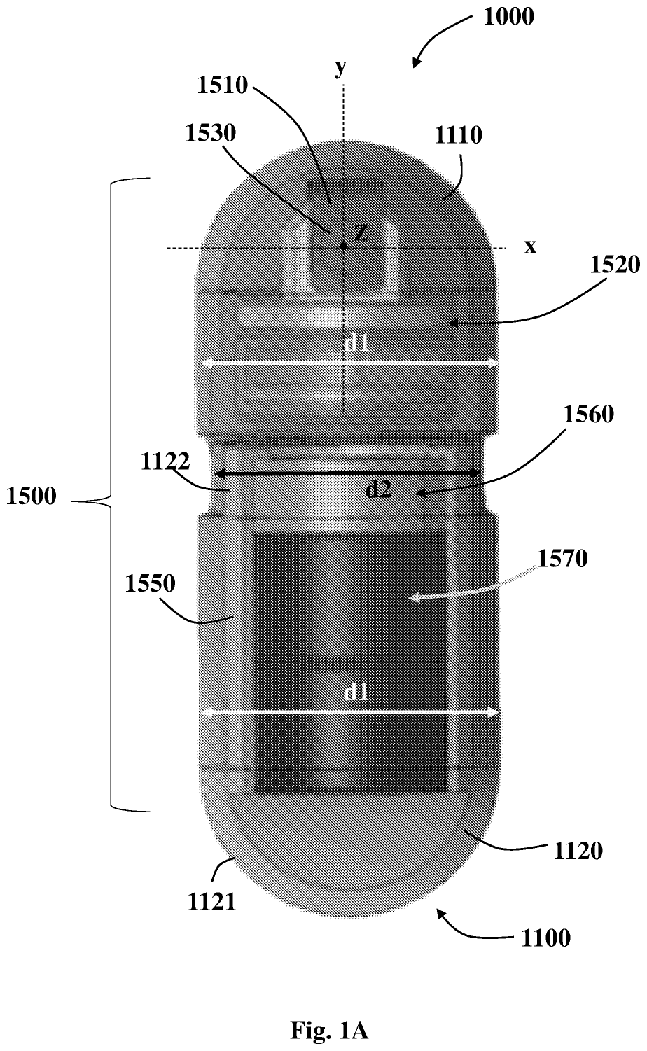

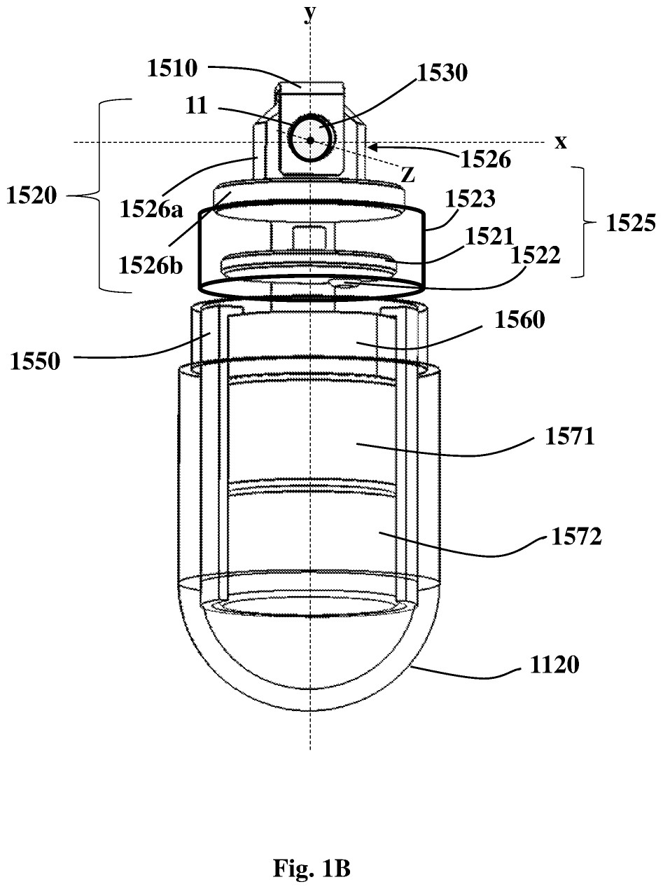

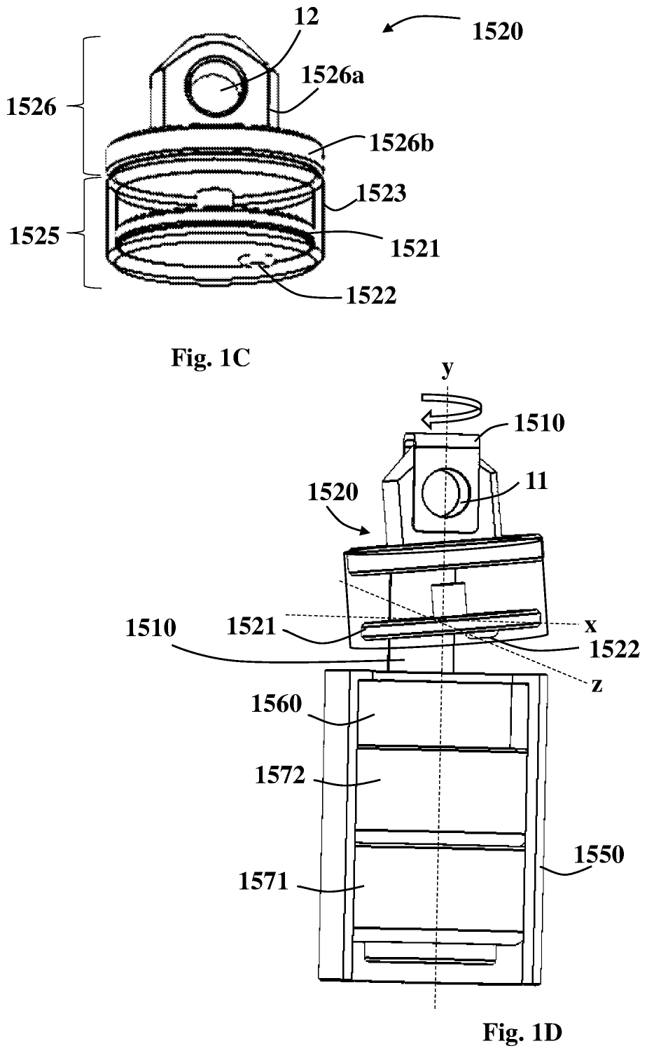

Reference is made to FIGS. 1A-1D, schematically illustrating an ICD 1000, for stimulating a GIT of a subject, according to some embodiments. The ICD 1000 may include at least:
(i) a capsule-housing 1100 including two parts: a 1^{st} capsule member 1110 and a second capsule member 1120, where the 1^{st} capsule member may be shaped as a semi-sphere or a semi-ellipsoid having a maximal diameter “d1” and the 2^{nd} capsule member 1120 may have a first part 1121 having the same maximal diameter “d1” and a second part 1122 having a different maximal diameter “d2” that is smaller than “d1”: d1>d2, to allow insertion (e.g. by interlocking) of the second part 1122 into the 2^{nd} capsule member 1120, as shown in FIG. 1A; and
(ii) a motion device 1500 located entirely within the capsule-hosing 1100.

The ICD 1000 may have an elongated cigar-shape of a cylinder having rounded edges symmetrically arranged around a rotation axis “y”.

According to some embodiments, as shown in FIGS. 1B-1C, the motion device 1500 may include:
a fixture element 1510, which may be rigid bent strap having a fixture-opening 11;
a weight assembly 1520, having a weight-opening 12;
a hinge 1530 configured for being threaded/inserted through both the fixture-opening 11 and through the weight-opening 12 through a swinging axis “x”, for hinging the weight assembly 1520 to the fixture element 1510 for enabling a swinging pendulum like movement about the swinging axis “x” of the weight assembly 1520 with respect to the fixture element 1510;
a control unit 1560;
a power supply 1570 including batteries 1571 and 1572; and
a holder 1550 connected to the fixture element 1510 for affixing the fixture element 1510 thereto and hold the power supply 1570 and the control unit 1560 thereby.

According to some embodiments, as shown in detail in FIGS. 1B and 1C, the weight assembly 1520 may include at least:
(a) a motor set 1525 combining the weight and the motor in a single packaged set, the motor set 1525 including for example: (i) a rotatable element 1521 such as a rotatable disc, rotatable about a rotation axis “y”, which is perpendicular to the swinging axis “x”; (ii) at least one inner eccentric (off-axis with respect to rotation axis “y”) weight element 1522; and (iii) at least one motor 1523 rotating the rotatable element 1521 and the eccentric weight element 1522 connected thereto, about the rotation axis “y”; and
(b) a connector such as a reversed T-shaped connector 1526 configured to connect at one side thereof to a side of the motor set 1525 (e.g., to an upper external side of the rotating element 1521) and to movably connect to the fixture element 1510 via the hinge 1530 at another side thereof.

According to some embodiments, the weight element 1522 may be integrally connected to a side of the rotatable element 1521 e.g., by forming both elements 1521-1522 as a single piece concentrating more material in an off-axis area that forms the eccentric weight element 1522.

As illustrated in FIG. 1D, once the rotatable element 1521 is rotated (by the motor 1523) about the rotation axis “y”, the eccentric weight element 1522 rotates along with it and causes changes in the center of mass location with respect to the rotation axis “y” thereby causes the entire weight assembly 1520 to swing with respect to the swinging axis “x”.

The hinged movable connection between the fixture element 1510 and the weight assembly 1520 forces the weight assembly 1520 to mainly move in a swinging pendulum-like movement about the swinging axis “x”. However, sudden and random changes in the power/current supply causing sudden changes in the swings amplitude may cause the weight assembly 1520 to slightly divert its force and apply ponding forces at different direction, which may cause the entire ICD 1000 to move in different directions thereby applying pounding forces or stimuli to different parts of the GIT section's inner wall.

According to some embodiments, as shown in FIG. 1C, the connector 1526 may include a first connector part 1526a having the weight-opening 12 thereover and a second connector part 1526b, integrally connected to the first connector part 1526a. The second connector part 1526b may have a surface area at one side thereof, that is similar in shape and dimensions to the dimensions and the shape of an external side of the motor 1523 to which it connects. The first connector part 1526a may be movably connectable to the fixture element 1510 via the hinge 1530, by having the openings 11 and 12 of the fixture-element 1510 and of the first connector part 1526a coaxially aligned and having the hinge 1530 inserted or threaded through the aligned openings 11 and 12.

FIG. 1D shows the weight assembly 1520 in a full swing state in which it is fully tilted to one side.

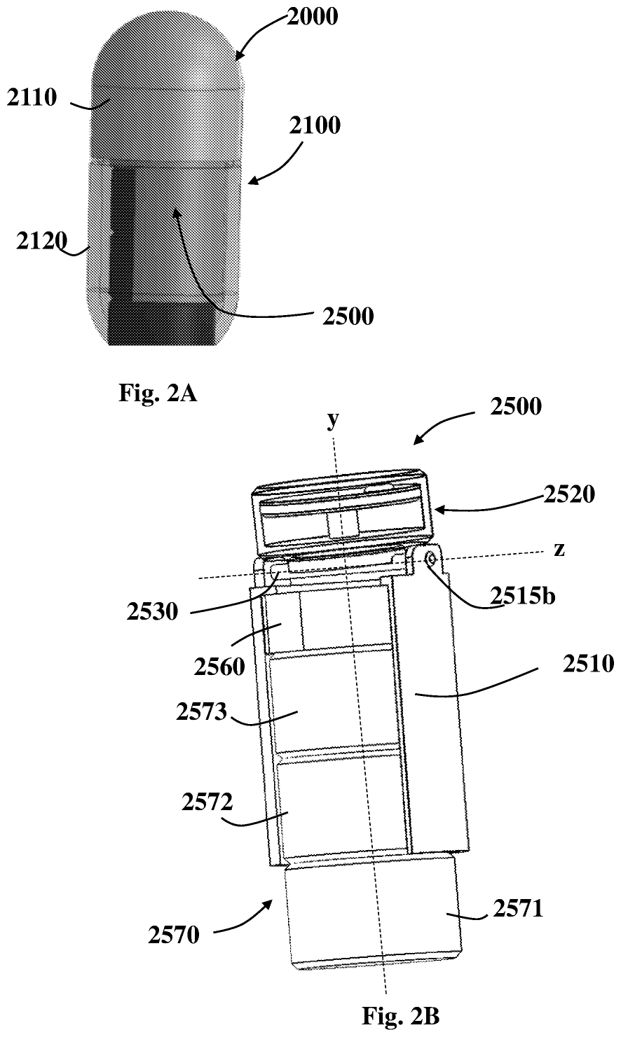

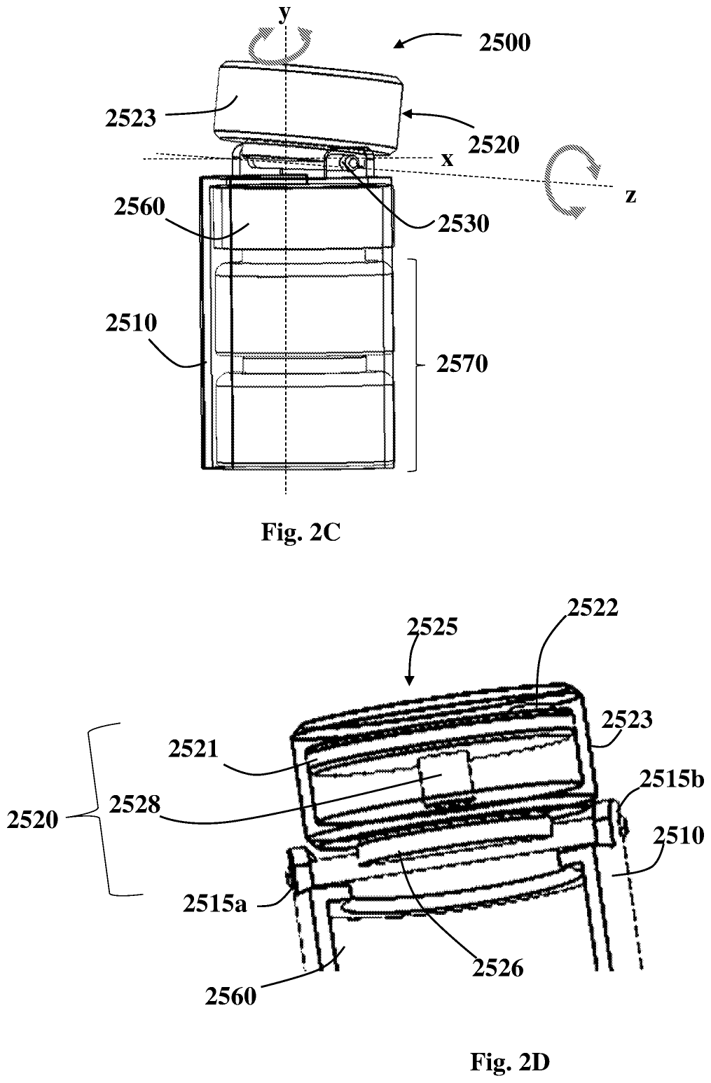

Reference is now made to FIGS. 2A-2D, schematically illustrating an ICD 2000, according to other embodiments. The ICD 2000 may comprise at least:
(i) a capsule-housing 2100 including two parts: a 1^{st} capsule member 2110 and a second capsule member 2120; and
(ii) a motion device 2500 located entirely within the capsule-hosing 2100.

According to some embodiments, as shown in FIGS. 2B-2D, the motion device 2500 may include:
a fixture element 2510, having two holes 2515a and 2515b at one side of the fixture element 2510;
a weight assembly 2520;
a hinge 2530 connected to the weight assembly 2520 such that the hinge 2530 edges are inserted through the holes 2515a and 2515b of the fixture element 2510 in a rotatable manner that enables rotation of the hinge 2530 (connected to the weight assembly 2520) about the swinging axis “x”;
a control unit 2560; and
a power supply 2570 including batteries.

In this case, the fixture element 2510 is also used as a holder for holding the power supply 2570 and the control unit 2560.

As shown in FIG. 2D, the weight assembly 2520 may include at least:
(a) a motor set 2525 combining the weight and the motor in a single packaged set, the motor set 2525 including for example: (i) a rotatable element 2521 such as a rotatable disc, rotatable about a rotation axis “y”, which is perpendicular to the swinging axis “x”; (ii) at least one inner eccentric (off-axis with respect to rotation axis “y”) weight element 2522; and (iii) at least one motor 2523 rotating the rotatable element 2521 and the eccentric weight element 2522 connected thereto, about the rotation axis “y”; and
(b) a connector 2526 such as a disc or plate connector 1526 configured to connect at one side thereof to a side of the motor set 2525 and to the hinge 2530 via another side thereof.

The weight assembly 2520 may form a solid single unit rotatable around the swinging axis “x”, with relation to the fixture element 2510.

According to some embodiments, and similarly to the swinging principles discussed with relation to motion device 1000 of FIGS. 1A-1D, the swinging motion about the swinging axis “x” is made by rotating of the rotatable element 2521 of the motor set 2525 and the eccentric weight element 2522 connected thereto, about the rotation axis “y”.

According to some embodiments, as shown in FIG. 2D, the rotatable element 2521 may be rotated by the rotation of the motor 2523 by connecting thereto via a connecting member 2528.

FIG. 2C shows the weight assembly 2520 in a full swing state in which it is fully tilted to one side.

According to some embodiments, there is provided a motion device having a rotating motor similar to motors 1523 and 2523 which is a rotatably cylinder forming a volume of space between the rotating cylinder and the rotatable element, where the rotatable element and the cylinder motor form a rotatable cylindrical box. In this configuration, at least one weight element may be freely placed within the formed enclosed rotatable cylindrical box, e.g., to mechanically form random pounding forces, caused by the free movement of the free weight element inside the cylindrical rotatable box. The un-connected free weight element may be placed in addition to an eccentric weight element or instead the attached eccentric element.

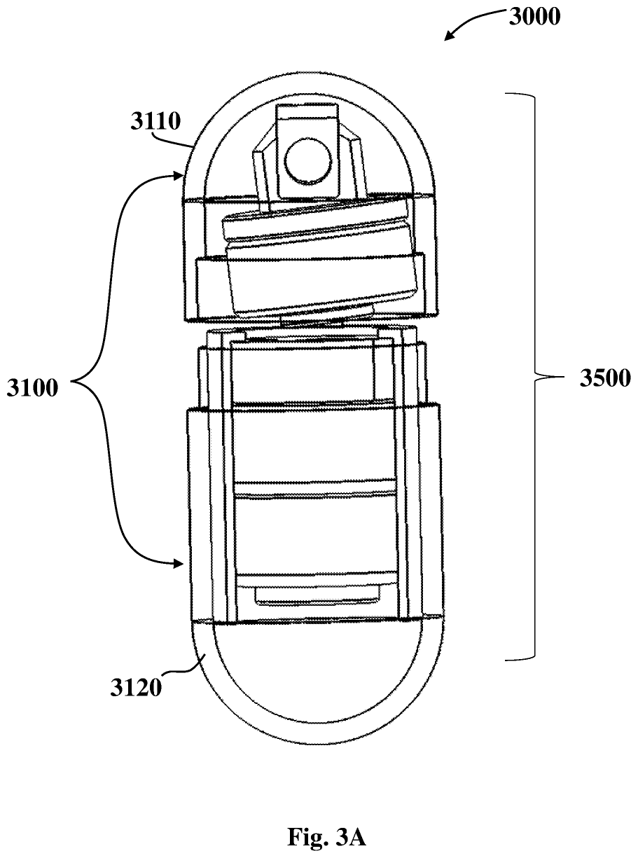

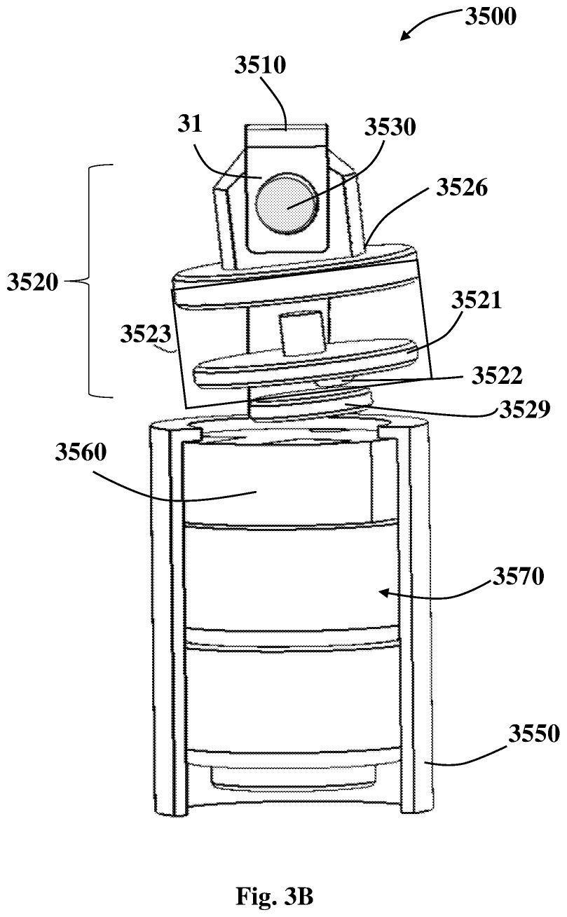

Reference is made to FIGS. 3A-3B, schematically illustrating an ICD 3000, for stimulating a GIT of a subject having an additional weight element, according to other embodiments.

The ICD 3000 may have a similar swinging design as that of ICD 1000 and may include at least:
(i) a capsule-housing 3100 including two parts: a 1^{st} capsule member 3110 and a second capsule member 3120; and
(ii) a motion device 3500 located entirely within the capsule-hosing 1100.

According to some embodiments, as shown in FIG. 3B, the motion device 3500 may include:
a fixture element 3510, which may be rigid bent strap having a fixture-opening;
a weight assembly 3520, having a weight-opening 32;
a hinge 3530 configured for being threaded/inserted through both the fixture-opening 31 and through the weight-opening through a swinging axis “x”, for hinging the weight assembly 3520 to the fixture element 1510 for enabling a swinging pendulum like movement about the swinging axis “x” of the weight assembly 1520 with respect to the fixture element 3510;
a control unit 3560;
a power supply 3570; and
a holder 3550 connected to the fixture element 3510 for affixing the fixture element 3510 thereto and hold the power supply 3570 and the control unit 1560 thereby.

According to some embodiments, as shown in detail in FIG. 3B, the weight assembly 3520 may include at least:
(a) a motor set 3525 combining the weight and the motor in a single packaged set, the motor set 3525 including for example: (i) a rotatable element 3521 such as a rotatable disc, rotatable about a rotation axis “y”, which is perpendicular to the swinging axis “x”; (ii) at least one inner eccentric (off-axis with respect to rotation axis “y”) weight element 3522; and (iii) at least one motor 3523 rotating the rotatable element 1521 and the eccentric weight element 3522 connected thereto, about the rotation axis “y”;
(b) a connector such as a reversed T-shaped connector 3526 configured to connect at one side thereof to a side of the motor set 3525 (e.g., to an upper external side of the rotating element 3521) and to movably connect to the fixture element 3510 via the hinge 1530 at another side thereof; and
an additional external weight element 3529 e.g., located centrally with respect to the rotation axis “y”, attached to an external side of the motor set 3525.

According to some embodiments, the additional weight element 3529 may add weight to the overall weight of the weight assembly 3520 optionally in a symmetrical manner with respect to the rotation axis “y” thereby adding more momentum to the swinging movement of the weight assembly 3520.

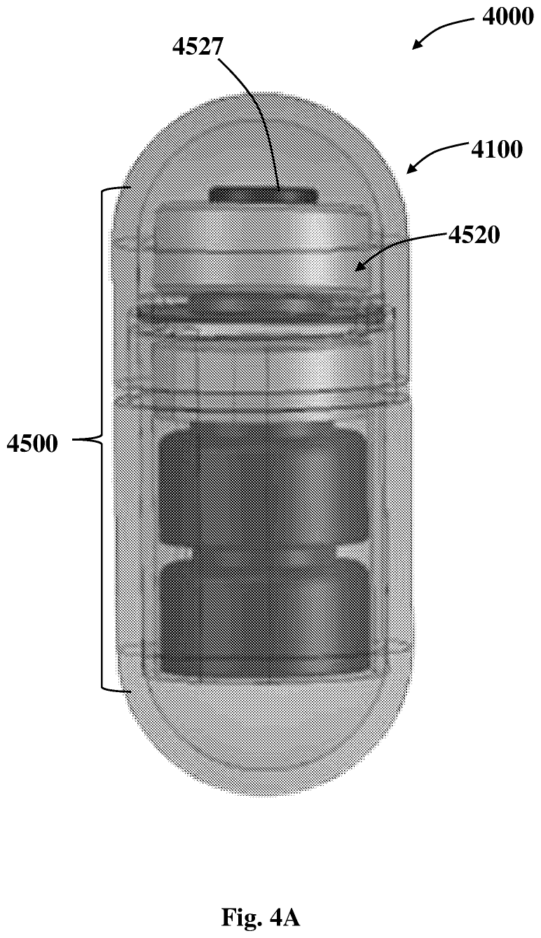

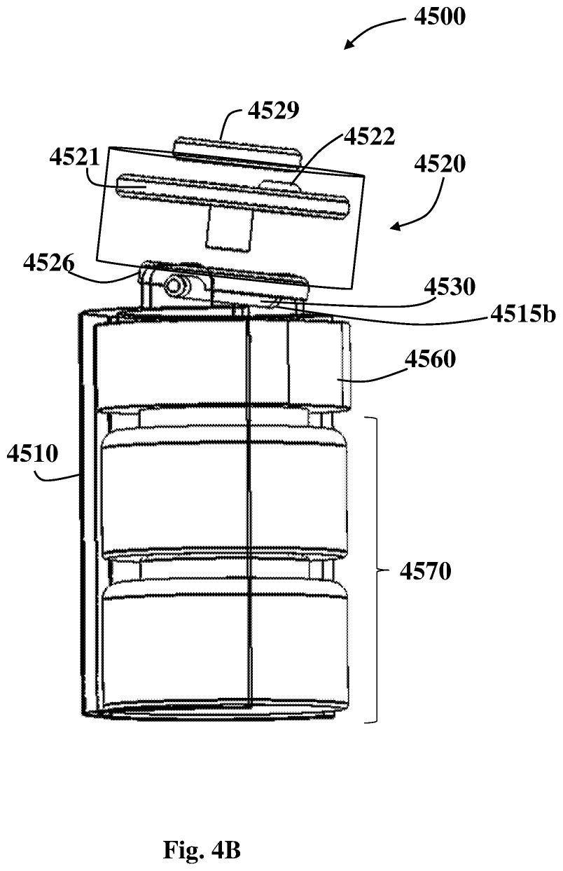

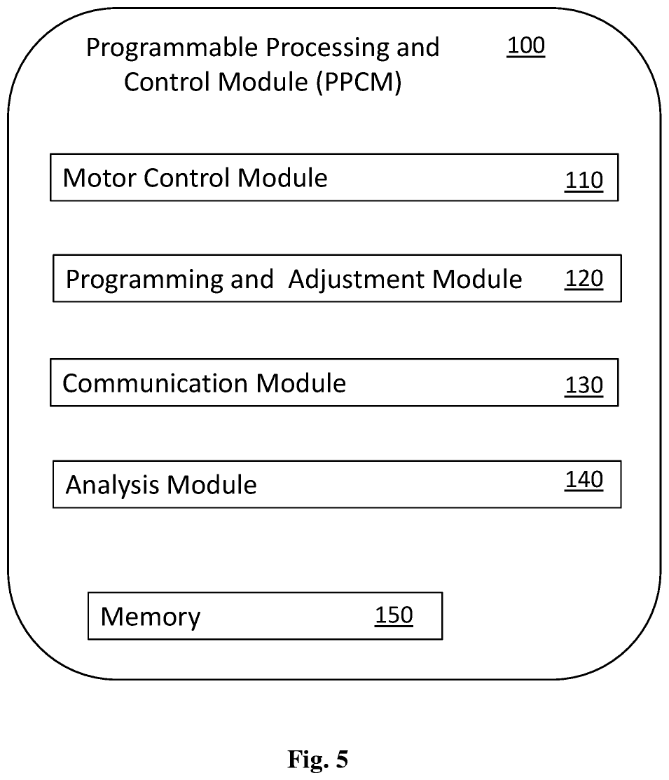

Reference is now made to FIGS. 4A-4B, schematically illustrating an ICD 4000, according to other embodiments. The ICD 4000 may comprise at least:
(i) a capsule-housing 4100 including two parts: a 1^{st} capsule member 4110 and a second capsule member 4120; and
(ii) a motion device 4500 located entirely within the capsule-hosing 4100.

According to some embodiments, as shown in FIGS. 4B-4B, the motion device 4500 may be similar to the motion device 2500 and may include:
a fixture element 4510, having two holes at one side of the fixture element 4510;
a weight assembly 4520;
a hinge 4530 connected to the weight assembly 2520 such that the hinge 4530 edges are inserted through the holes of the fixture element 4510 in a rotatable manner that enables rotation of the hinge 4530 (connected to the weight assembly 4520) about the swinging axis “x”;
a control unit 4560; and
a power supply 4570 including batteries.

In this case, the fixture element 4510 is also used as a holder for holding the power supply 4570 and the control unit 4560.

As shown in FIG. 4B, the weight assembly 4520 may include at least:
(a) a motor set, combining the weight and the motor in a single packaged set, the motor set including for example: (i) a rotatable element 4521 such as a rotatable disc, rotatable about a rotation axis “y”, which is perpendicular to the swinging axis “x”; (ii) at least one inner eccentric (off-axis with respect to rotation axis “y”) weight element 4522; and (iii) at least one motor 4523 rotating the rotatable element 2521 and the eccentric weight element 4522 connected thereto, about the rotation axis “y”;
(b) a connector 4526 such as a disc or plate connector 1526 configured to connect at one side thereof to a side of the motor set 2525 and to the hinge 2530 via another side thereof; and
an additional external weight element 4529 e.g., located centrally with respect to the rotation axis “y”, attached to an external side of the motor set 4525.

According to some embodiments, the additional weight element 4529 may add weight to the overall weight of the weight assembly 4520 optionally in a symmetrical manner with respect to the rotation axis “y” thereby adding more momentum to the swinging movement of the weight assembly 4520.

According to other embodiments, the additional weight(s) may also be eccentric located at a same relative position with respect to the rotation axis “y” as the internal weight element 1522 and/or 2522.

Aspects of embodiments pertain to a motion device to be used inside a capsule-housing to form an ICD, which can be one or more of:
remotely controlled;
programmable and adjustable for various treatment purposes;
controllable based on analysis/processing of sensor data/signals received from external sensor(s) or from sensor(s) embedded in the motion device or in the ICD.

According to some embodiments a control unit of each motion device or of each ICD such as control units 1560, 2560, 3560 and/or 4560, may include a programmable processing and control unit (PPCU) such as PPCU 100.

According to embodiments, the PPCU 100 may include at least:
a motor control module 110 configured at least to control operation of the at least one motor of the motion device for controlling swinging motion characteristics of the weight assembly of the motion device (of the ICD) such as: swinging span, swinging/pounding intensity/amplitude, swinging frequency, break time-intervals characteristics such as breaks duration and operation duration, random operation control etc.;
a programming and adjustment module 120, for enabling programming and reprogramming/adjusting control program(s) according to which the motor(s) of the specific motion device or ICD is (are) controlled;
a communication module 130 configured for communication with external devices such as for transmitting sensed information/signals to an external computing device for external offline or real time analysis and/or with internal instruments, such as sensors, the programming and adjustment module 120 and/or the motor control module 110; and
(optionally) an analysis module 140 for receiving signals/data from other instruments of the ICD or external instruments such as sensors, analyzing/processing received data/signals and store the received and analyze data via a memory;
a memory unit 150 for retrievable storage of data such as received data, control commands and plans etc.

According to some embodiments, the motor control module may be configured to control one or more motors and/or one or more motions devices of the specific ICD by using software and/or hardware means such as by using a designated software/hardware designed for controlling the motor(s) based on one or more of:
one or more preset control modes, each mode being associated with a different set of control commands;
one or more activation codes/signal, each code/signal associated with a different control mode.

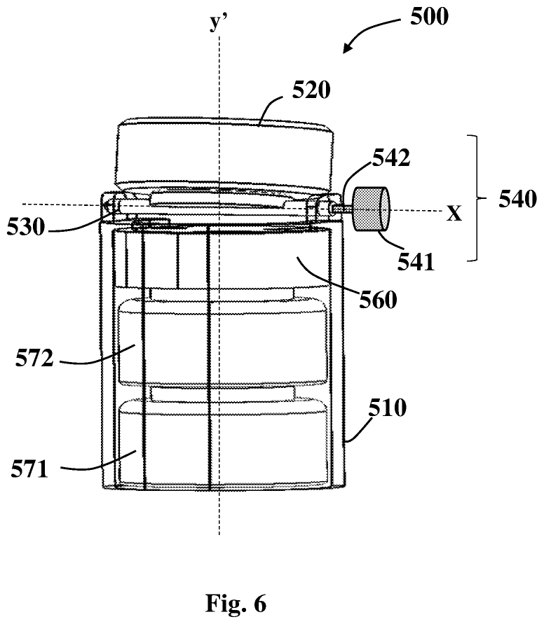

Reference is now made to FIG. 6, schematically illustrating a motion device 500 for an ICD, where the motion device 500 is based on direct hinge 530 rotation, according to other embodiments. The motion device 500 may include at least:
a fixture element 510;
a weight element 520;
a hinge 530;
a motor set 540 including a motor 541 and a drive shaft 542 that is fixedly connected to the hinge 530 and rotatable by the motor 541;
a control unit 560; and
a power supply including one or more batteries such as batteries 571 and 572.

The weight element 520 may be fixedly connected to the hinge 530, where the hinge 530 may be movably (pivotally) connected to the fixture element 510. The motor 541 may be configured to rotate the drive shaft 542 in various rotation speeds and in both clockwise and counterclockwise directions. In these embodiments, the weight element's 520 center of mass may be symmetrical with respect to a main axis “y′”.

The hinge 530 defines a swing axis “x” about which the weight element 520 causes the swinging motion for pounding against the capsule housing (not shown) for GI stimulation.

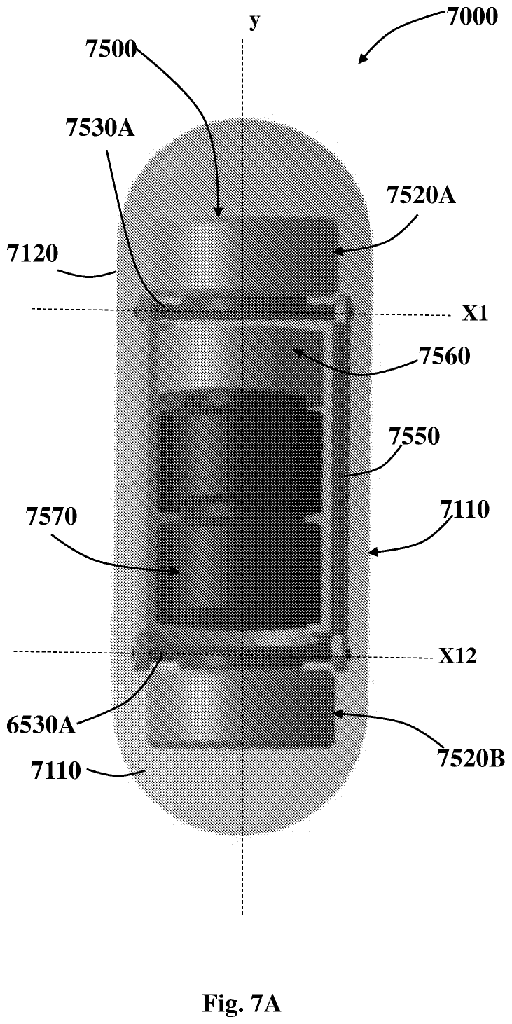

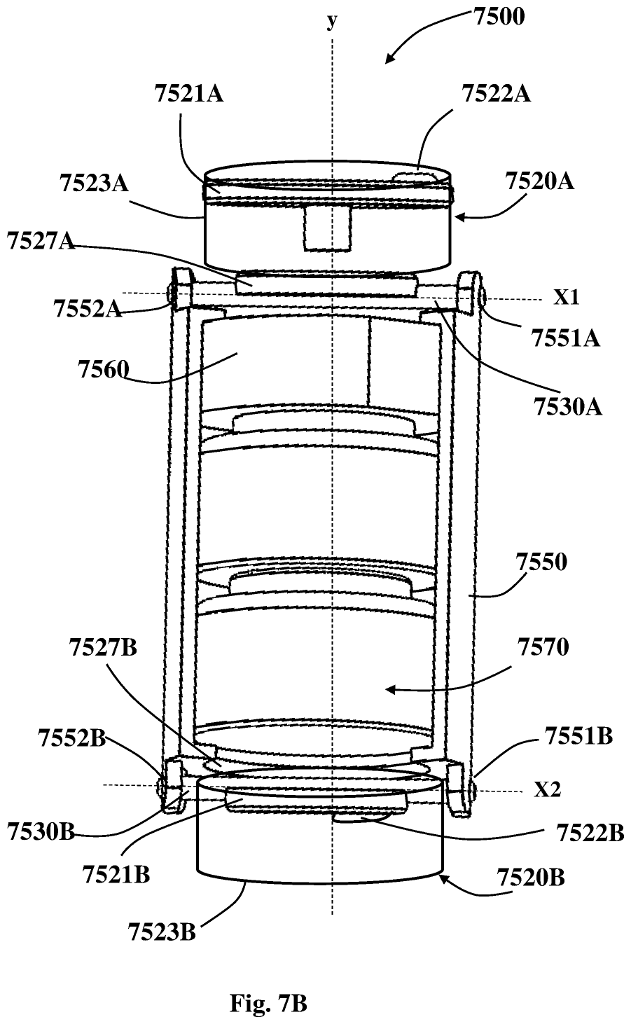

Reference is now made to FIGS. 7A and 7B, schematically illustrating an ICD 7000 having a motion device 7500 with two weight assemblies 7520A and 7520B, each having its own rotating motor set, where the two motor sets 7540A and 7540B are coaxially positioned with respect to one another along their mutual rotation axis “y”, according to some embodiments. The ICD 7000 may include at least:

a capsule-housing 7100 including a 1^{st} casing member 7110 and a second capsule member 7120 connectable or engageable with one another in a sealable manner to form the capsule-housing 7100;
a motion device 7500 having:
(i) a first weight assembly 7520A;
(ii) a second weight assembly 7520B;
(iii) a first hinge 7530A;
(iv) a second hinge 7530B;
(v) a control unit 7560;
(vi) a power supply 7570 with one or more batteries; and
(vii) a fixture element 7550 serving both as a casing for the control unit 7560 and power supply 7570 and as a fixture for the weight assemblies 7620A and 7620B.

The weight assemblies 7520A and 7520B may be coaxially arranged such that the rotation axis “y” of the motors (embedded in the weight assemblies 7520A and 7520B) is the same, and such that the perpendicular swinging axes “x1” and “x2”, respectively, are parallel.

According to some embodiments, each weight assembly 7520A/7520B includes:
a rotating element 7521A/7521B;
at least one weight element such as weight element 7522A/7522B, each connected to a respective rotating element 7521A/7521B; and
a rotating motor 7523A/7523B, configured to rotate therein the rotating element 7521A/7521B and the weight element 7522A/7522B for causing the swinging motion of the entire weight assembly 7520A/7520B about its respective swinging axis x1/x2.

According to some embodiments, the fixture element 7550 of each weight assembly 7520A/7520B, respectively, may be movably (rotatably) connected to a different edge of the fixture element 7550, where each weight assembly 7520A/7520B may be fixedly connected to its respective hinge 7530A/7530B e.g., via a connector such as a connector plate 7527A/7527B.

According to some embodiments, each of the edges of the fixture element 7550 may have two holes for receiving therethrough the respective hinge 7530A/7530B: a first edge approximated to the first weight assembly 7520A may have two holes 7551A and 7552A, receiving therethrough the first hinge 7530A, and a second edge approximated to the second weight assembly 7520AB may have two holes 7551B and 7552B, receiving therethrough the second hinge 7530B.

The weight elements 7522A and 7522B may be both eccentric with respect to the rotation axis “y” and optionally be of the same of different weight.

According to some embodiments, the control unit 7560 may be configured to control both weight assemblies 7520A and 7520B in one or more operation modes such as by one or more of:
coordinating simultaneous rotation of both motors 7523A and 7523B in opposite or same rotation direction, different or same rotation speeds and/or power for creating similar pounding force application from both sides of the motion device 7500, etc.;
applying different operation modes for each weight assembly 7520A/7520B, e.g., for applying different forces and/or different swinging frequencies at each side of the ICD 7000;
interchangeably or plan-based switching of activation between the two weight assemblies 7520A/7520B by turning one off while turning the other on in a preset operation time-span for each weight assembly 7520A/7520B or arbitrarily;

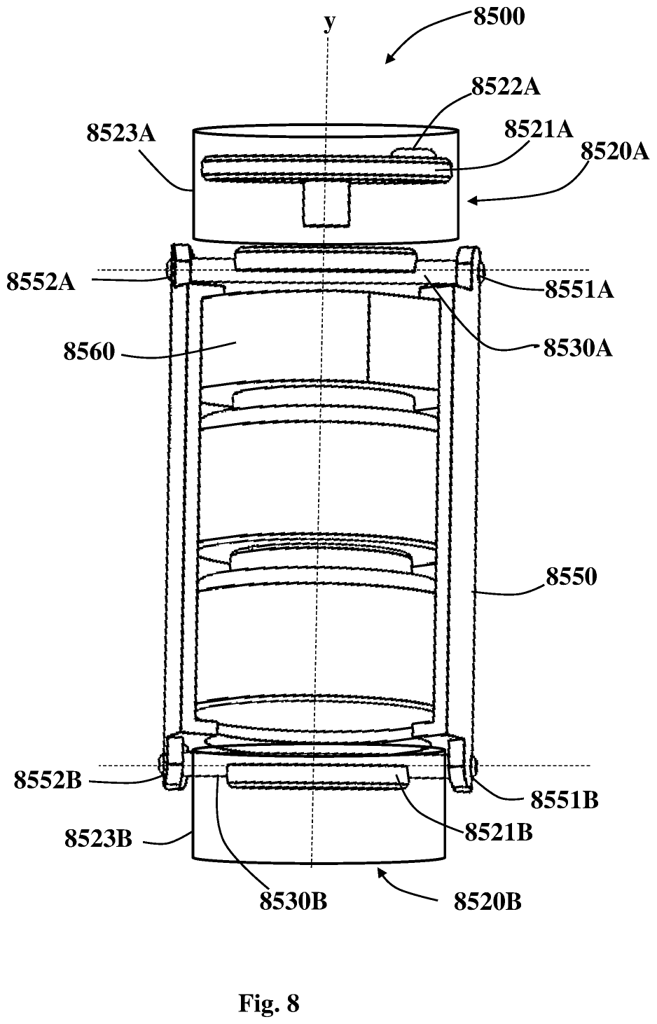

Reference is now made to FIG. 8, schematically illustrating a motion device 8500 for an ICD having two weight assemblies 8520A and 8520B, coaxially arranged with relation to a rotation axis “y” of motors 8523A/8523B of the weight assemblies 7520A/7520B, respectively, according to some embodiments.

According to these embodiments, the motion device 8500 includes:
(i) a first weight assembly 8520A;
(ii) a second weight assembly 8520B;
(iii) a first hinge 8530A;
(iv) a second hinge 8530B;
(v) a control unit 8560;
(vi) a power supply 8570 with one or more batteries; and
(vii) a fixture element 8550 serving both as a casing for the control unit 8560 and power supply 8570 and as a fixture for the weight assemblies 8620A and 8620B.

The weight assemblies 8520A and 8520B may be coaxially arranged such that the rotation axis “y” of the motors (embedded in the weight assemblies 8520A and 8520B) is the same, and such that the perpendicular swinging axes “x1” and “x2”, respectively, are parallel.

According to some embodiments, the first weight assembly 8520A may include:
a rotating element 8521A, symmetrically arranged around the rotation axis “y”;
at least one weight element such as weight element 8522A, connected to a respective rotating element 8521A and located eccentrically from the rotation axis “y”; and
a rotating motor 8523A, configured to rotate therein the rotating element 8521A and the weight element 8522A for causing the swinging motion of the entire weight assembly 8520A about its respective swinging axis “x1”.

According to some embodiments, the second weight assembly 8520B may include:
a rotating element 8521B, such as a rotatable circular disc, symmetrically arranged around the rotation axis “y” and
a rotating motor 8523B, configured to rotate therein the rotating element 8521B and the weight element 8522B.

According to some embodiments, since the second weight assembly 8520B does not include an eccentric weight element, and is symmetrically arranged about the rotation axis “y”, its swinging motions about its swinging axis “x2” are random and mostly much smaller in intensity and/or amplitude than those of the first swinging assembly 8520A thereby can be used, for example, to dramatically reduce pounding forces applied when the first weight assembly 8520A is deactivated. This may enable upon activation of the entire ICD of the motions device 8500, to begin activation of the ICD prior to the swallowing thereof by the subject, and begin with an initial time span in which only the second weight assembly 8520B is activated, e.g., for preventing the subject from feeling any movement of the ICD when swallowing the ICD and while it is passed through the subject's upper parts of the GIT such as the mouth, the esophagus and begin a more intense pounding by activating the first weight assembly 8520A after the initial time-span in which the ICD is estimated to have reached one of: the stomach, the small intestines or the large intestines. The initial time-span may be set according to the GTI section in which the ICD should be used for GIT stimuli, depending on medical requirements associated with the specific subject.

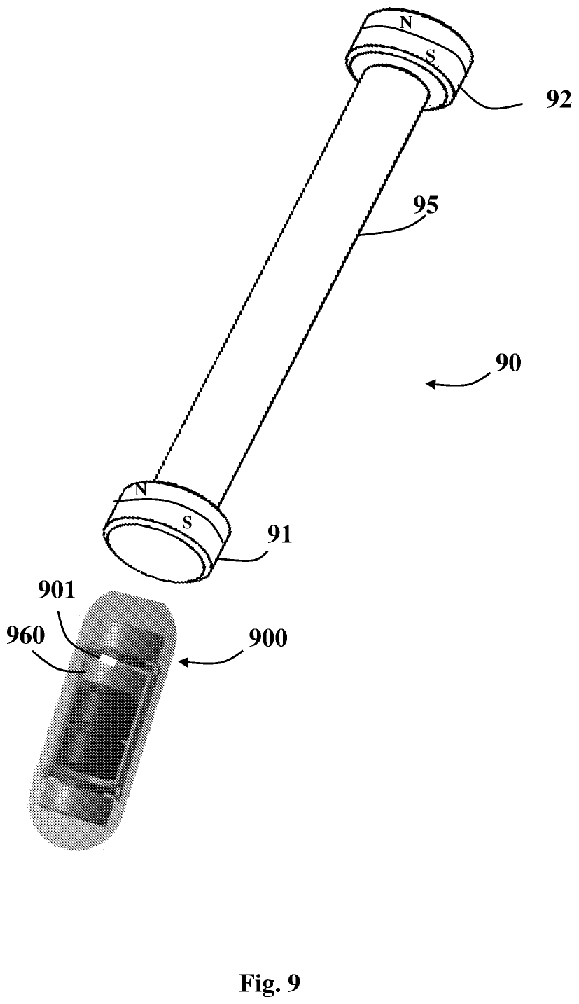

Reference is now made to FIG. 9, schematically illustrating an ICD 900 including at least one magnetic sensor 901, for identification of one or more magnetic activation sequences, according to some embodiments, by use of an external magnetic instrument 90 having two magnetic poles 91 and 92 of opposite charges, for applying sequences of positive and negative magnetic fields to the ICD 900 sensible by the at least one magnetic sensor 901.

As shown in FIG. 9, the external magnetic instrument 90 may include two permanent magnets 91 and 92 each attached to a different edge of a holding stick 95, where one (first) magnet 91 is positioned such that a south S side thereof faces away from the edge of the stick 95 and the other (second) magnet 92 is positioned such that a north N side thereof faces away from the edge of the stick 95 creating a bipolar magnet instrument 90 for enabling manual applying of a magnetic activation sequence by flipping the sides of the magnetic instrument 90 according to a preset required activation sequence such as N-S-N-S within a predefined limited activation time-span (such as ranging between 15-40 seconds).

According to some embodiments, the magnetic sensor 901 may be configured to sense magnetic force/field and output a signal indicative of the N/S sequence. The signal outputted by the magnetic sensor 901 may then be received by the control unit 960 of the ICD 900, which identifies the N/S sequence and enables activation of the ICD 900 upon identification of a sequence that matches one or more of preset identifiable activation sequences. The control unit 960 is also configured to identify whether the sequence of signals received is within the limited activation time span. Therefore, the control unit 960 may be configured to only activate (turn on) the ICD 900 if a permitted sequence is identified and if the permitted sequence identified was delivered within the predefined activation time span.

According to some embodiments, the at least one magnetic sensor 901 is located in or on the control unit 960.

According to some embodiments, the control unit 960 may be configured such that it has several permitted magnetic sequences stored in a memory unit thereof, where each sequence may be associated with a different activation mode/plan for enabling using the same type and configuration of the ICD 900 for different operation ways such as to enable adapting the right GIT stimulation treatment to the specific patient.

According to some embodiments, the magnetic instrument used may be a simple permanent magnet with two poles N and S located at 180 degrees from one another.

According to other embodiments, the magnetic instrument may include at least one coiled electric wire, where the S/N induced magnetic field can be changeable in a controllable manner by controlling current direction through the coiled electric wire. In this way the overall magnetic force applied may be controllable as well.

Aspects of disclosed embodiments pertain to an activation method for activating an ICD by using a magnetic instrument that can generate or used to generate a magnetic sequence for ICD activation, The activation method may include, for example:
(i) manually or automatically applying a magnetic sequence of positive or negative magnetic fields to at least one magnetic sensor of the ICD within a specific actual time span;
(ii) sensing magnetic forces/fields using at least one magnetic sensor;
(iii) receiving output signals from the at least one magnetic sensor indicative of the applied magnetic sequence;
(iv) identifying a sequence of output signals from the at least one sensor;
(v) checking whether the received output signals within a predefined limited activation time span matches a sequence stored in the ICD;
(vi) if such match is identified optionally, identify an operation mode that is associated with the identified sequence; and
activating the ICD optionally according to the identified associated operation mode.

Aspects of disclosed embodiments pertain to implementations of the ICD in any design of the ICD described above for improving an indirect effect of the ICD, for example, over livestock animals' milk production, for example, by designing the size, activation timings, applied force levels, etc. to generate the pounding in the animal's stomach.

For example, for improving milk production and/or milk quality extracted from cows, the ICD may be designed to significantly stimulate one of the cow's stomachs.

### EXAMPLES

Example 1 is an ingestible capsule device (ICD) comprising at least: a capsule-housing; and at least one motion device comprising at least:
a fixture element;
at least one motor;
at least one weight assembly movable by use of the at least one motor, wherein the weight assembly is movably connected, via a hinge, to the fixture element to allow pendulum-like swinging movement of the weight assembly, with respect to the fixture element, about a swinging axis “x”, wherein the swinging movement about the swinging axis “x” causes pounding of the weight of the weight assembly on at least one inner wall of the capsule-housing; and
a power supply for powering the at least one motor;
wherein the motion device is located inside the capsule-housing, and
wherein the motion device is configured to stimulate a gastrointestinal tract (GTI) section of the subject, by transmitting pounding forces of the weight assembly from the inner wall of the capsule-housing onto an inner wall of the GIT section.
In example 2, the subject matter of example 1 may include, wherein the weight assembly comprises: at least one weight element, a rotatable element configured to be rotated by the at least one motor setup, around a rotation axis “y”, which is perpendicular to the swinging axis “x”, wherein the at least one weight element is connected to the rotatable element in an off-axis location on the rotatable element, with respect to the rotation axis “y”, and wherein the off-axis location of the at least one weight element causes changes in the center of mass with respect to the swinging axis “x”, and wherein the pendulum-like swinging movement about the swinging axis “x” is generated by the rotation of the rotatable element and the at least one off-axis weight element connected thereto around the rotation axis “y”.
In example 3, the subject matter of example 2, wherein the rotatable element comprises a rotatable circular disc, symmetrically arranged around the rotation axis “y”.
In example 4, the subject matter of any one or more of examples 1 to 3, wherein the ICD may further comprise a control unit configured at least for controlling operation of the at least one motor.
In example 5, the subject matter of example 4 may include, wherein the control unit comprises one or more of: a printed circuit board (PCB), a control chip, an integrated circuit device; one or more switching devices, one or more sensors, one or more transmitters, one or more receivers, one or more processors, a micro-electro-mechanical system (MEMS), wherein the maximal length of the at least one motor setup and/or the at least one weight assembly is 10 mm.
In example 6, the subject matter of example 5 may include, wherein the control unit is further configured to receive operation command signals and operate the at least one motor setup according to received operation command signals.
In example 7, the subject matter of any one or more of examples 5 to 6 may include, wherein the ICD further comprises at least one sensor for sensing one or more of:
one or more characteristics of the ingestible capsule device;
time from ingestible capsule device intake;
location of the ingestible capsule device;
one or more characteristics of an environment of the ingestible capsule device.
In example 8, the subject matter of example 7 may include, wherein the control unit further comprises a programmable processing and control unit (PPCU), configured to one or more of: control operation of the at least one motor based on a programmable operation plan; receive sensor data from the at least one sensor; analyze the received sensor data to determine one or more characteristics of the ingestible capsule device and/or an environment thereof; control operation of the ingestible capsule device, based on determined one or more characteristics of the ingestible capsule device and/or an environment of the ingestible capsule device.
In example 9, the subject matter of any one or more of examples 1 to 8 may include, wherein the ingestible capsule device is operated by controlling electrical current/power supply to each of the at least one motor.
In example 10, the subject matter of example 9 may include, wherein the controlling of the at least one motor comprises one or more of:
intermittently changing motor setup current/power supply for intermittent change in motor setup activation and/or operation mode;
activating the at least one motor setup at a specific timing after the at least motor setup is turned on enabling activation after an activation delay period for allowing the ingestible capsule device to pass specific sections of the GIT before being activated;
remote and/or external activation and operation control via a remote control device controllable and operable by a user;
generating occasion random activation of the at least one motor;
generating random changes in activation power of the at least one motor.
In example 11, the subject matter of any one or more of examples 1 to 10, wherein the capsule-housing comprises two sealably connectable or interlocking parts: a 1^{st} capsule-member and a 2^{nd} capsule member.
In example 12, the subject matter of example 11 may include, wherein the 1^{st} capsule member is configured to cover one of the at least one weight assembly and the 2^{nd} pounding member is configured to cover other parts and/or components of the motion device.
In example 13, the subject matter of any one or more of examples 1 to 12, ay include, wherein the at least one motor comprises at least one of: an electrical motor, a micro direct current (DC) motor, a micro alternating current (AC) motor, a cylindrical rotating micro motor.
In example 14, the subject matter of any one or more of examples 1 to 13 may include, wherein the weight assembly comprises at least two weight elements: an internal weight element embedded within the at least one motor setup and at least one external weight element configured to add an additional weight to the weight of the weight assembly.
In example 15, the subject matter of any one or more of examples 1 to 14 may include, wherein the at least one motion device further comprises a holder connected to the fixture element for affixing the fixture element thereto and hold the power supply and/or a control unit thereby.
In example 16, the subject matter of any one or more of examples 1 to 14 may include, wherein the at least one motion device further comprises a holder configured to hold thereby at least one of: the at least one power supply and/or the control unit.
In example 17, the subject matter of any one or more of examples 1 to 14 may include, wherein the fixture element of the motion device includes two holes or a channel through which the hinge is movably inserted and wherein the weight assembly fixedly connects to the hinge, such that the swinging movement is enabled by rotation of the connected weight assembly and hinge inside the holes or the channel of the fixture element.
In example 18, the subject matter of example 1 may include, wherein the weight assembly comprises a single weight rotatably connected via the hinge to the fixture element, wherein the at least one motor setup comprises an electrical motor setup rotating a drive shaft connected to the hinge or being part of the hinge.
In example 19, the subject matter of example 1 may include 1, wherein the at least one motion device comprises several motion devices and/or several weight assemblies, all located and operated within the same capsule-housing, for applying pounding forces via different areas of the capsule-housing.
In example 20, the subject matter of example 19 may include, wherein each weight assembly is operated by a separate motor using the same or separate power supplies.
In example 21, the subject matter of example 20 may include, wherein swinging axes of the several weight assemblies are: all parallel to one another; all angular in a non-parallel manner to one another; or one or more of the several swinging axes being parallel to one or more swinging axes of one or more other weight assemblies.
In example 22, the subject matter of any one or more of examples 1 to 21 may include, wherein the at least one weight assembly connects to inner wall of the capsule-housing via an elastic connecting element enabling retraction of the weight assembly to a relaxed state.
In example 23, the subject matter of any one or more of examples 1 to 22 may include, wherein at least one part of the capsule-housing which engages the at least one weight assembly when pounding thereover, is made from a flexible or elastic material for enhancing transmission of pounding forces from the capsule-housing's inner wall onto the inner wall of the subject's the GIT section.
In example 24, the subject matter of any one or more of examples 1 to 23 may include, wherein at least one part of the capsule-housing which engages the at least one weight assembly is made from a material of a lower thickness than thickness of one or more other parts of the capsule-housing that do not engage with the at least one weight assembly.
In example 25, the subject matter of any one or more of examples 1 to 24 may include, wherein the ICD may further comprise at least one magnetic sensor for sensing magnetic fields and/or forces, wherein the ingestible capsule device has a control unit configured to receive signals outputted by the at least one magnetic sensor, identify a magnetic sequence within a predefined activation time span and activate the ingestible capsule device upon identification of a permitted magnetic sequence applied within the activation time span.
In example 26, the subject matter of example 25 may include, wherein the application of magnetic fields is carried out by use of a magnetic instrument configured for applying a magnetic sequence by applying a sequence of positive or negative magnetic fields.
In example 27, the subject matter of any one or more of examples 1 to 27 may include, wherein the at least one weight assembly comprises at least two weight assemblies, each operated by a different motor, each weight assembly movably connects to the fixture element via a different hinge defining a different swinging axis.
In example 28, the subject matter of example 27 may include, wherein the swinging axis of at least one of the at least two weight assemblies is parallel to the swinging axis of at least one other of the at least two weight assemblies.
In example 29, the subject matter of any one or more of examples 27 to 28 may include, wherein the swinging axis of at least one of the at least two weight assemblies is angular to the swinging axis of at least one other of the at least two weight assemblies.
Example 30 is motion device for an ingestible capsule having a capsule-housing, the motion device comprising at least:
a fixture element; at least one motor;
at least one weight assembly, movable by use of the motor, wherein the weight assembly is movably connected, via a hinge, to the fixture element to allow pendulum-like swinging movement of the weight assembly, with respect to the fixture element, about a swinging axis “x”, wherein the swinging movement about the swinging axis “x” causes pounding of the weight of the weight assembly on an inner wall of the capsule-housing; and
a power supply for powering the at least one motor;
wherein the motion device is configured to be placed in an ingestible capsule device for stimulation of a GTI section of the subject.
In example 31, the subject matter of example 30 may include, wherein the at least one of the at least one weight assembly comprises a weight element and a rotatable element configured to be rotated by the motor setup, around a rotation axis “y”, which is angular or perpendicular to the swinging axis “x”, wherein the rotatable element is rotatable by the at least one motor, wherein the at least one weight element is connected to the rotatable element in an off-axis location on the rotatable element, with respect to the rotation axis “y”, wherein the off-axis location of the at least one weight element causes changes in the center of mass with respect to the swinging axis “x”, and wherein the pendulum-like swinging movement about the swinging axis “x” is generated by the rotation of the rotatable element and the at least one off-axis weight element connected thereto, around the rotation axis “y”.
In example 32, the subject matter of example 31 may include, wherein the rotatable element and the weight element are both embedded in the motor setup as part of the weight assembly.
In example 33, the subject matter of example 30 may include, wherein the fixture element has a fixture-opening for receiving the hinge therethrough and the weight assembly has a connector having a connector-opening for receiving the hinge therethrough, wherein the fixture-opening and the connector-opening are coaxially arranged with respect to the swinging axis “x”.
In example 34, the subject matter of example 30 may include, wherein the fixture element has two holes through which the hinge is threaded, wherein the hinge is fixedly connected to the at least one weight assembly.
In example 35, the subject matter of any one of examples 30 to 34 may include, wherein the at least one weight assembly further comprises at least one additional external weight element connected to an external side of the weight assembly, eccentrically or centrically with respect to the rotation axis “y”, at least for adding weight to the total weight of the weight assembly.
In example 36, the subject matter of any one or more of examples 30 to 35, wherein the motion device may further comprise an elastic connecting element connecting at one end thereof to the weight assembly and at another end thereof to inner wall of the capsule-housing, for enabling retraction of the weight assembly to a relaxed state during the swinging movement of the weight assembly.
In example 37, the subject matter of example 36, wherein the elastic connecting element comprises a coiled spring.
In example 38, the subject matter of any one or more of examples 30 to 37 may include, wherein the motion device further comprises at least one control unit configured at least for controlling activation and/or operation of the motion device.
In example 39, the subject matter of example 38 may include, wherein the control unit is further configured to one or more of:
control operation of the at least one motor based on one or more programmable operation plans;
receive sensor data from the at least one sensor that is embedded in the ingestible capsule device or external;
analyze the received sensor data to determine one or more characteristics of the ingestible capsule device and/or an environment thereof;
control operation of the ingestible capsule device, based on determined one or more characteristics of the ingestible capsule device and/or an environment of the ingestible capsule device;
control operation of the motion device based on received code, magnetic or signal sequence.
In example 40 the subject matter of any one or more of examples 30 to 39 may include, wherein the motion device further comprises at least one magnetic sensor for sensing magnetic fields and/or forces, wherein a control unit of the ingestible capsule device or of the motion device is configured to receive signals outputted by the at least one magnetic sensor, identify a magnetic sequence within a predefined activation time span and activate the ingestible capsule device upon identification of a permitted magnetic sequence applied within the activation time span.
In example 41, the subject matter of example 40 may include, wherein the application of magnetic fields is carried out by use of a magnetic instrument configured for applying a magnetic sequence by applying a sequence of positive or negative magnetic fields.
In example 42, the subject matter of any one or more of examples 30 to 41 may include, wherein the at least one weight assembly comprises at least two weight assemblies, each operated by a different motor, each weight assembly movably connects to the fixture element via a different hinge defining a different swinging axis.
In example 43, the subject matter of example 42 may include, wherein the swinging axis of at least one of the at least two weight assemblies is angular or parallel to the swinging axis of at least one other of the at least two weight assemblies.
In example 44, the subject matter of any one or more of examples 30 to 43 may include, wherein at least one weight assembly comprises two weight assemblies each moveably connecting to the fixture element via a different hinge, defining two different swinging axes that are parallel or angular to one another: a first weight assembly comprising at least a rotatable element, a weight element and a motor, wherein the motor is configured to rotate the rotatable element about a rotation axis “y”, and wherein the weight element is eccentric with relation to the rotation axis enabling thereby the swinging of the weight element with respect to its swinging axis “x1”; and a second weight assembly comprising at least a rotatable element and a motor configured to rotate the rotatable element about the rotation axis “y”.
In example 45, the subject matter of example 44 may include, wherein the second weight assembly also comprises one or more weight elements, located symmetrically or eccentrically with respect to the rotation axis “y”.
Example 46 is an activation method for activation of an ingestible capsule device (ICD), the ICD comprising a motor-based mechanism, for pounding inner walls of a gastrointestinal tract (GIT) of a subject, the ICD having a control unit and at least one magnetic sensor, the method comprising at least:
(i) applying a magnetic sequence of positive or negative magnetic fields to at least one magnetic sensor of the ICD within a specific actual time span;
(ii) sensing magnetic forces/fields using at least one magnetic sensor;
(iii) receiving output signals from the at least one magnetic sensor indicative of the applied magnetic sequence;
(iv) identifying a sequence of output signals from the at least one sensor;
(v) checking whether the received output signals within a predefined limited activation time span matches a sequence stored in the ICD;
(vi) if such match is identified optionally, identify an operation mode that is associated with the identified sequence; and
(vii) activating the ICD optionally according to the identified associated operation mode.
In example 47, the subject matter of example 46 may include, wherein the applying of a magnetic sequence is carried out manually by using a bipolar magnetic instrument.

Although the above description discloses a limited number of exemplary embodiments of the invention, these embodiments should not apply any limitation to the scope of the invention, but rather be considered as exemplifications of some of the manners in which the invention can be implemented.

The systems, modules, units, devices, apparatuses etc. or parts thereof, may be programmed to perform particular functions pursuant to computer readable and executable instructions, rules, conditions etc. from programmable hardware and/or software based execution modules that may implement one or more methods or processes disclosed herein, and therefore can, in effect, be considered as disclosing a “special purpose computer” particular to embodiments of each disclosed method/process.

Computer/Processor readable and executable instructions or signals may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

In the above disclosure, unless otherwise stated, terms such as “substantially”, “about,” approximately, etc., that specify a condition or relationship characterizing a feature or features of an embodiment of the invention, are to be understood to mean that the condition or characteristic is defined to within tolerances that are acceptable for operation of the embodiment for an application for which it is intended.

It is important to note that the methods/processes and/or systems/devices/subsystems/apparatuses etc., disclosed in the above Specification, are not to be limited strictly to flowcharts and/or diagrams provided in the Drawings. For example, a method may include additional or fewer processes or steps in comparison to what is described in the figures. In addition, embodiments of the method are not necessarily limited to the chronological order as illustrated and described herein.

Terms used in the singular shall also include a plural scope, except where expressly otherwise stated or where the context otherwise requires.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

Unless otherwise stated, the use of the expression “and/or” between the last two members of a list of options for selection indicates that a selection of one or more of the listed options is appropriate and may be made i.e., enabling all possible combinations of one or more of the specified options. Further, the use of the expression “and/or” may be used interchangeably with the expressions “at least one of the following,” “any one of the following” or “one or more of the following,” followed by a listing of the various options.

It is appreciated that certain features of the invention, which are, for clarity, described in the context of separate embodiments or example, may also be provided in combination in a single embodiment. Conversely, various features of the invention, which are, for brevity, described in the context of a single embodiment, example and/or option, may also be provided separately or in any suitable sub-combination or as suitable in any other described embodiment, example or option of the invention. Certain features described in the context of various embodiments, examples and/or optional implementation are not to be considered essential features of those embodiments, unless the embodiment, example and/or optional implementation is inoperative without those elements.

It is noted that the terms “in some embodiments,” “according to some embodiments,” “according to some embodiments of the invention”, “for example,” “e.g.,” “for instance” and “optionally” may herein be used interchangeably.

The number of elements shown in the Figures should by no means be construed as limiting and is for illustrative purposes only.

It is noted that the terms “operable to” can encompass the meaning of the term “modified or configured to.” In other words, a machine “operable to” perform a task can in some embodiments, embrace a mere capability (e.g., “modified”) to perform the function and, in some other embodiments, a machine that is actually made (e.g., “configured”) to perform the function.

Throughout this application, various embodiments may be presented and/or may relate to a range format. It should be understood that a description in a range format is merely for convenience and brevity and should not be construed as an inflexible limitation on the scope of the embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

1. An ingestible capsule device comprising:
(a) a capsule housing; and
(b) at least one motion device, located inside the capsule housing, comprising:
a fixture element;
a motor
a power supply powering the motor;
a weight assembly comprising the motor and an eccentric weight element rotated by the motor, wherein the weight assembly is connected, via a hinge, to the fixture element, to allow pendulum-like swinging movement of the weight assembly, with respect to the fixture element, about the hinge, wherein rotation of the eccentric weight causes the swinging movement of the weight assembly about the hinge, and wherein the weight assembly, when swinging, pounds the capsule housing to stimulate a gastrointestinal tract (GTI).

2. The ingestible capsule device of claim 1, wherein the weight assembly further comprises: a rotatable element configured to be rotated by the motor, around a rotation axis “y”, which is perpendicular to the hinge, and wherein the eccentric weight element is connected to the rotatable element in an off-axis location.

3.-4. (canceled)

5. The ingestible capsule device of claim 1, wherein the motor weight assembly is 4-10 mm in length.

6. The ingestible capsule device of claim 1, further comprising a control unit configured at least for controlling operation of the motor, according to received operation command signals, wherein controlling motor operation comprises one or more of: controlling operation based on a programmable operation plan; receiving sensor data from at least one sensor; analyzing the received sensor data to determine one or more characteristics of the ingestible capsule device and/or an environment thereof; and controlling the operation based on the characteristics of the ingestible capsule device and/or the environment of the ingestible capsule device.

7. The ingestible capsule device of claim 1, further comprising at least one sensor for sensing one or more of: one or more characteristics of the ingestible capsule device; a time from ingestible capsule device intake; a location of the ingestible capsule device; and one or more characteristics of an environment of the ingestible capsule device.

8. (canceled)

9. The ingestible capsule device of claim 1, wherein the pendulum-like swinging movement of the weight assembly is adjustable by adjusting the supplied power frequency, duration, and power level.

10. The ingestible capsule device of claim 9, wherein the control of the motor comprises one or more of:
intermittently changing the motor current to make intermittent changes in the motor operation mode;
activating the motor after an activation delay period;
remotely activating the motor via a remote control device controllable and operable by a user;
generating occasional random activation;
generating random changes in the activation power level.

11.-12. (canceled)

13. The ingestible capsule device of claim 1, wherein the motor comprises at least one of: an electrical motor, a micro direct current (DC) motor, a micro alternating current (AC) motor, a cylindrical rotating micro motor.

14. The ingestible capsule device of claim 1, wherein the weight assembly comprises at least two weight elements: the eccentric weight element rotated by the motor setup and at least one additional weight adding momentum to the swinging movement of the weight assembly.

15.-18. (canceled)

19. The ingestible capsule device of claim 1, wherein the at least one motion device comprises several motion devices and/or several weight assemblies, all located and operated within the same capsule housing, for applying pounding forces via different areas of the capsule housing.

20. The ingestible capsule device of claim 19, wherein each weight assembly is operated by a separate motor using the same or separate power supplies.

21. The ingestible capsule device of claim 20, wherein swinging axes of the several weight assemblies are:
all parallel to one another;
all angular in a non-parallel manner to one another; or
one or more of the several swinging axes being are parallel to one or more swinging axes of one or more other weight assemblies.

22. The ingestible capsule device of claim 1, wherein the weight assembly pounds an inner wall of the capsule housing.

23. The ingestible capsule device of claim 1, wherein at least one part of the capsule housing that engages the weight assembly when pounding thereover, is made from a flexible or elastic material, for enhancing transmission of pounding forces from the capsule housing to the GIT.

24. The ingestible capsule device of claim 1, wherein at least one part of the capsule housing that engages the weight assembly is made from a material of a lower thickness than a thickness of one or more other parts of the capsule housing that do not engage with the weight assembly.

25. The ingestible capsule device of claim 1, further comprising at least one magnetic sensor for sensing magnetic fields, wherein the ingestible capsule device has a control unit configured to receive signals outputted by the at least one magnetic sensor, identify a magnetic sequence within a predefined activation time span and activate the ingestible capsule device upon identification of a permitted magnetic sequence applied within the activation time span.

26. (canceled)

27. The ingestible capsule device of claim 1, wherein the at least one weight assembly comprises at least two weight assemblies, each operated by a different motor, each weight assembly movably connects to the fixture element via a different hinge defining a different swinging axis.

28.-29. (canceled)

30. A motion device for an ingestible capsule having a capsule housing, the motion device comprising at least:
a fixture element;
a motor and a power supply powering the motor;
a weight assembly, comprising the motor and an eccentric weight element rotated by the motor, wherein the weight assembly is connected, via a hinge, to the fixture element to allow pendulum-like swinging movement of the weight assembly, with respect to the fixture element, about the hinge, wherein rotation of the eccentric weight causes the swinging movement about the hinge, such that the swinging weight of the weight assembly pounds the capsule housing to stimulate a gastrointestinal tract.

31.-34. (canceled)

35. The motion device of claim 30, wherein the weight assembly comprises at least one additional weight element for adding momentum to the swinging movement of the weight assembly.

36-39. (canceled)

40. The motion device of claim 30, further comprising at least one magnetic sensor for sensing magnetic fields and a control unit, wherein the control unit is configured to receive signals outputted by the at least one magnetic sensor, identify a magnetic sequence within a predefined activation time span and activate the motion device upon identification of a permitted magnetic sequence applied within the activation time span.

41.-47. (canceled)
